(19) United States
(12) Patent Application Publication
(10) Pub. No.: US 2007/0208074 A1
(43) Pub. Date: Sep. 6, 2007

(54) Methods and compositions for treating and preventing tumors

(76) Inventors: Azad M. Bonni, Brookline, MA (US); Nuria de la Iglesia, Brookline, MA (US); Genevieve Konopka, Los Angeles, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/657,965

(22) Filed: Jan. 24, 2007

Related U.S. Application Data
(60) Provisional application No. 60/762,033, filed on Jan. 24, 2006.

(51) Int. Cl. A61K 31/337

(52) U.S. Cl. 514/449

(57) ABSTRACT

The present invention provides methods for reducing the growth or invasiveness of tumors.

### RELATED APPLICATIONS

This application claims priority to provisional patent application Ser. No. 60/762,033, filed on Jan. 24, 2006, the entire contents of which is hereby incorporated by reference.

### BACKGROUND OF THE INVENTION

As the most malignant primary central nervous systems tumors, high grade anaplastic astrocytoma and glioblastoma multiforme respond poorly to contemporary multimodality treatment programs employing surgical resection, radiation therapy and chemotherapy with a median survival of less than one year after initial diagnosis. The development of effective new agents and novel treatment modalities against these very poor prognosis brain tumors are urgently needed.

### SUMMARY OF THE INVENTION

The present invention provides a method of reducing the growth or invasiveness of a tumor cell by contacting a tumor cell with an agent that reduces the level or activity of IL-8 or the IL-8 receptor (IL-8R), or alternatively, an agent that increases the level or activity of STAT3. The tumor cell is characterized as having a Phosphatase and Tensin Homolog (PTEN) deficiency or loss. PTEN-deficient refers to at least 5%, 10%, 25%, 50%, 75%, 90% or greater reduction and up to a total loss of PTEN expression compared to a normal wild type cell of a given tissue type. For example, the tumor cell is of a central nervous system (CNS) tissue. The tumor is a primary neural tumor or is the result of metastasis from outside the nervous system. Either one of these two agents may also be used to reduce or prevent the growth or invasiveness of a brain tumor (e.g., glioma such as an astrocytoma (e.g., glioblastoma multiforme)) in a mammal. Optionally, the agent that reduces the level or activity of IL-8 is used in combination with an agent that increases the level or activity of STAT3. In all foregoing aspects of the invention, the agent that reduces the level or activity of IL-8 or IL-8R or that increases the level or activity of STAT 3 is a small molecule inhibitor, such as a CXCRX antagonist (e.g., CXCR1 antagonist such as repertaxin or CXCR2 antagonist). Small molecules such as repertaxin and SB drugs (e.g., SB455821, SB265610 from Glaxo SmithKline Beecham) are IL8 receptor antagonists, which are useful for treatment of a class of cancers characterized by PTEN deficiency or loss.

A small molecule inhibitor is a compound that is less than 2000 daltons in mass. The molecular mass of the inhibitory compounds is preferably less than 1000 daltons, more preferably less than 600 daltons, e.g., the compound is less than 500 daltons, 400 daltons, 300 daltons, 200 daltons, or 100 daltons. Preferably, the inhibitor is not a peptide or proteinaceous in nature. If desired, a second therapeutic regimen may also be used in the present invention including, for example, surgery (tumor resection), chemotherapy, or radiotherapy.

By “reduce the expression or activity of IL-8 or IL-8R” is meant to reduce the level or biological activity of IL-8 or IL-8R relative to the level or biological activity of IL-8 or IL-8R in an untreated control. The level or activity is preferably reduced by at least 10%, 20%, 30%, 40%, 50%, 60%, 70%, 80%, 90%, or 100%, relative to an untreated control. Since STAT3 represses IL-8 expression, a reduction in the biological activity of IL-8 is, for example, an increase in the expression or activity of STAT3. For example, expression or activity of STAT3 is increased by at least 10%, 20%, 30%, 40%, 50%, 60%, 70%, 80%, 90%, 100%, or even greater than 100%, relative to an untreated control, thereby reducing IL-8R signaling and ultimately reducing the growth and invasiveness of the glioma. As used herein, the term “activity” with respect to a IL-8R polypeptide includes any activity which is inherent to the naturally occurring IL-8R protein. Thus, the term “IL-8R activity” includes any activities of molecules involved in IL-8R signaling, such as binding to IL-8.

By “treating or preventing a brain tumor” is meant ameliorating any of the conditions or symptoms associated with the brain tumor (e.g., glioma) before or after it has occurred including, for example, seizures, headaches, limb paresis, swelling of the ankles and legs, fever, chills, stiff neck, abdominal pain, unusual bruising, nausea, vomiting, and skin rash. Alternatively, alleviating a symptom of a brain tumor may involve reducing the tumor size (e.g., by reducing the proliferation of tumor cells or by increasing the rate of apoptosis of tumor cells) or reducing the rate of tumor metastasis relative to an untreated control. As compared with an equivalent untreated control, such reduction or degree of prevention is at least 5%, 10%, 20%, 40%, 50%, 60%, 80%, 90%, 95%, or 100% as measured by any standard technique. A patient who is being treated for a brain tumor is one who a medical practitioner has diagnosed as having such a condition. Diagnosis may be by any suitable means. Diagnosis and monitoring may involve, for example, detecting the level of brain tumor in a biological sample (e.g., tissue biopsy, blood test, or urine test), detecting the level of a surrogate marker (e.g., PTEN, bcl-2, and p53) of the brain tumor in a biological sample, detecting symptoms associated with a brain tumor, or detecting tumor cells. A patient in whom the development of a brain tumor is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

As used herein, by “IL-8R” is meant a polypeptide that forms a complex with the IL-8 ligand and is involved in various signaling pathways involving, for example, STAT3. The IL-8R proteins of the invention are substantially identical to the naturally occurring IL-8R (e.g., accession numbers AAL31314, NP_{—}001548, and NP_{—}000625, the sequences of which are hereby incorporated by reference). According to this invention, brain tumors are treated or prevented when IL-8R activity or expression is reduced by at least 5%, 10%, 20%, 30%, 40%, 50%, 60%, 70%, 80%, 90%, or 100% below control levels as measured by any standard method (e.g. Northern blot analysis).

By a “IL-8R gene” is meant a nucleic acid that encodes an IL-8R protein.

By “IL-8R fusion gene” is meant an IL-8R promoter and/or all or part of an IL-8R coding region operably linked to a second, heterologous nucleic acid sequence. In preferred embodiments, the second, heterologous nucleic acid sequence is a reporter gene, that is, a gene whose expression may be assayed; reporter genes include, without limitation, those encoding glucuronidase (GUS), luciferase, chloramphenicol transacetylase (CAT), green fluorescent protein (GFP), alkaline phosphatase, and beta-galactosidase.

By “purified antibody” is meant antibody which is at least 60%, by weight, free from proteins and naturally occurring organic molecules with which it is naturally associated. Preferably, the preparation is at least 75%, more preferably 90%, and most preferably at least 99%, by weight, antibody, e.g., an IL-8 or IL-8R specific antibody (e.g., CXC chemokine receptor-1 (CXCR1) or -2 (CXCR2) antibodies). A purified antibody may be obtained, for example, by affinity chromatography using recombinantly-produced protein or conserved motif peptides and standard techniques.

By “specifically binds” is meant an antibody that recognizes and binds an antigen or antigenic domain such as an IL-8R polypeptide but that does not substantially recognize and bind other non-antigen molecules in a sample, e.g., a biological sample, that naturally includes protein or domains of a target protein.

By “neutralizing antibodies” is meant antibodies that interfere with any of the biological activity of an IL-8 or IL-8R polypeptide (e.g., the ability to increase tumor cell proliferation). The neutralizing antibody may reduce IL-8R signaling activity by, preferably 50%, more preferably by 70%, and most preferably by 90% or more.

By “substantially identical,” when referring to a protein or polypeptide, is meant a protein or polypeptide exhibiting at least 75%, but preferably 85%, more preferably 90%, most preferably 95%, or even 99% identity to a reference amino acid sequence. For proteins or polypeptides, the length of comparison sequences will generally be at least 20 amino acids, preferably at least 30 amino acids, more preferably at least 40 amino acids, and most preferably 50 amino acids or the full length protein or polypeptide. Nucleic acids that encode such “substantially identical” proteins or polypeptides constitute an example of “substantially identical” nucleic acids; it is recognized that the nucleic acids include any sequence, due to the degeneracy of the genetic code, that encodes those proteins or polypeptides. In addition, a “substantially identical” nucleic acid sequence also includes a polynucleotide that hybridizes to a reference nucleic acid molecule under high stringency conditions.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., Current Protocols in Molecular Biology, John Wiley & Sons, New York, N.Y., 1998, hereby incorporated by reference.

By “substantially pure” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

The term “isolated DNA” is meant DNA that is free of the genes which, in the naturally occurring genome of the organism from which the given DNA is derived, flank the DNA. Thus, the term “isolated DNA” encompasses, for example, cDNA, cloned genomic DNA, and synthetic DNA.

By “an effective amount” is meant an amount of a compound, alone or in a combination, required to reduce or prevent hypertension or to reduce the growth or invasiveness of a brain tumor in a mammal. The effective amount of active compound(s) varies depending upon the route of administration, age, body weight, and general health of the subject. Ultimately, the attending physician or veterinarian will decide the appropriate amount and dosage regimen.

By a “candidate compound” is meant a chemical, be it naturally-occurring or artificially-derived. Candidate compounds may include, for example, peptides, polypeptides, synthetic organic molecules, naturally occurring organic molecules, nucleic acid molecules, peptide nucleic acid molecules, and components and derivatives thereof.

The term “pharmaceutical composition” is meant any composition, which contains at least one therapeutically or biologically active agent and is suitable for administration to the patient. Any of these formulations can be prepared by well-known and accepted methods of the art. See, for example, Remington: The Science and Practice of Pharmacy, 20^{th} edition, (ed. A. R. Gennaro), Mack Publishing Co., Easton, Pa., 2000.

The present invention provides significant advantages over standard therapies for treatment, prevention, and reduction, or alternatively, the alleviation of one or more symptoms associated with brain tumors. In addition, the candidate compound screening methods provided by this invention allow for the identification of novel therapeutics that modify the injury process, rather than merely mitigating the symptoms.

Cited publications including sequences defined by GENBANK™ accession numbers are incorporated herein by reference. Other features, objects, and advantages of the invention will be apparent from the description of the drawings.

### BRIEF DESCRIPTION OF THE FIGURES

FIG. 1A is a series of immunoblots showing the levels of LIFRβ, phosphorylated Tyr 705-STAT3 (PYS3), STAT3, PTEN, Akt, and Serine 473-phosphorylated Akt in lysates of mouse PTEN +/+ or PTEN −/− astrocytes.

FIG. 1B is a series of immunoblots showing the levels of PTEN, phosphorylated Akt, Akt, LIFRβ, Tyrosine 705-phosphorylated STAT3 (PYS3), STAT3, and actin in parental PTEN flox/flox astrocytes, PTEN −/− astrocytes, and murine embryonic fibroblasts (MEFs). The loss in PTEN expression was associated with an increase in phospho-Akt levels in both astrocytes and MEFs, and with a decrease in LIFRβ, PYS3 and total STAT3 levels in astrocytes but not MEFs. β-actin demonstrates equal loading.

FIG. 1C is a series of immunoblots showing the levels of PTEN, phosphorylated Akt, Akt, LIFRβ, PYS3, and STAT3 in serum-starved PTEN−/− astrocytes treated with the PI3K inhibitor LY294002 or DMSO vehicle control for 48 hours (left pannel). Asterisk indicates a non-specific band. The right panel shows immunoblotting of lysates from PTEN flox/flox astrocytes stably expressing a constitutively active form of Akt (caAkt). Activated Akt reduced LIFRβ levels.

FIG. 1D is a series of photographs representing immunocytochemical analysis of PTEN flox/flox and PTEN−/− astrocytes stained with an antibody to FOXO3. Nuclei were stained with a DNA dye (Hoechst). FOXO3 is excluded from the nucleus in PTEN−/− astrocytes.

FIG. 1E is a bar graph showing the levels of luciferase activity in PTEN flox/flox and PTEN−/− astrocytes. The transfected luciferase reporter gene is controlled by FOXO binding sites which has been transfected together with a Renilla expression plasmid serving as an internal control, and subjected to a dual luciferase assay. FOXO-dependent transcription is reduced in PTEN−/− astrocytes.

FIG. 1F is a photograph showing a chromatin immunoprecipitation analysis at the endogenous LIFRβ promoter in PTEN flox/flox and PTEN−/− astrocytes with a FOXO3 antibody. A rabbit anti-HA antibody was used as negative control. Negative controls for the PCR reaction were performed with primers for the E-cadherin promoter. FOXO3 occupied the endogenous LIFRβ promoter in PTEN flox/flox but not in PTEN−/− astrocytes.

FIG. 1G is a series of immunoblots showing the levels of LIFRβ, FOXO3, and actin in PTEN flox/flox and PTEN−/− astrocytes infected with a FOXO3 RNAi-encoding lentivirus (FOXOi) or an empty vector and selected with puromycin. The knockdown of endogenous FOXO3 results in downregulation of LIFRβ.

FIG. 1H is a series of immunoblots showing the levels of LIFRβ, PYS3, STAT3, and actin in PTEN−/− astrocytes transfected with a LIFRβ expression plasmid or a control vector that were left untreated or treated with LIF (L) for 15 minutes. LIFRβ restores LIF-induced STAT3 phosphorylation in PTEN −/− astrocytes.

FIG. 2A is a series of immunoblots showing the levels of PYS3 and STAT3 in glioma cells that harbor PTEN mutations (U87, A172, and U138) or that express wild type PTEN (SF188 and LN229) with the STAT3 and PYS3 antibodies. Cells were serum starved for 24 hours and then treated with 100 ng/ml CNTF (C) or 10 ng/ml LIF (L) for 15 minutes. LIF treatment robustly induced STAT3 tyrosine phosphorylation in the control neuroblastoma cell line SK-N-MC and in the wild type PTEN-expressing glioma cells, but failed to effectively induce the STAT3 phosphorylation in the PTEN-deficient glioma cells.

FIG. 2B is a series of graphs showing growth curves of PTEN-mutant glioma cell lines (U87 and A172) and PTEN-expressing (SF188) glioma cells lines that were treated with LIF (10 ng/ml daily) or left untreated. LIF significantly reduced SF188 cell population growth (n=3; paired t-test, asterisk indicates p<0.05).

FIG. 2C is a bar graph showing the population growth of SF188 cells infected with a retrovirus containing an IRES-GFP cassette alone or that also encodes wild type STAT3 or a dominant negative STAT3 (S3D). LIF (10 ng/ml daily) led to a significant reduction in population growth of control-vector or wild type STAT3-expressing cells (n=3; ANOVA,p<0.01), but not in S3D-expressing SF188 cells.

FIG. 2D is a pair of immmunoblots showing STAT3 and actin levels in 293T cells infected with either control lentivirus or lentivirus encoding one of two small interfering hairpin RNAs directed at STAT3 and selected with puromycin (left panel). The stat3i1 stat3i2 hairpin RNAs induced knockdown of endogenous human STAT3 with increasing effectiveness. The right panel is a graph showing population growth of LN229 cells infected with control lentivirus or lentivirus encoding stat3i1 or stat3i2 and selected with puromycin. LIF (10 ng/ml daily) led to a significant reduction in growth in control-infected (n=3; ANOVA, p<0.05), but not stat3i1 or stat 3i2 hairpin RNA-expressing LN229 cells.

FIG. 2E (left panel) is a series of immunoblots showing the levels of STAT1, STAT3, and actin in rat neural stem cells infected with either control lentivirus or lentivirus encoding the stat3i hairpin RNAs and selected with puromycin. Both hairpin RNAs induced knockdown of endogenous rat STAT3. STAT1 immunoblotting indicates that stat3i hairpin RNAs specifically reduce STAT3 but not STAT1 neural stem cells.

FIG. 2E (middle panel) shows a series of immunocytochemical photographs of rat neural stem cells infected with a control GFP-expressing lentivirus (panels a-d) or a GFP-expressing lentivirus also encoding stat3i2 (panels e-h). STAT3 immunostaining is reduced with STAT3 knockdown (panel f). Arrowheads indicate infected cells.

FIG. 2E (right panel) is a bar graph showing BrdU incorporation in rat neural stem cells treated with LIF (10 ng/ml, 48 hrs) following infection with lentivirus and selection with puromycin. Compared to no treatment, LIF treatment led to significantly less BrdU incorporation in vector-infected cells (n=3; ANOVA, p<0.05) but not in neural stem cells in which STAT3 was knocked-down.

FIG. 3A is a diagram of activated STAT3 (S3C) or a DNA-binding mutant version of S3C (mS3C) is shown in left panel. Middle panel: immunoblotting of lysates of 293T cells transfected with expression plasmids encoding FLAG-tagged S3C or mS3C or the control vector with the STAT3 antibody, an antibody to FLAG, or the β-actin antibody. Right panel shows relative luciferase activity of a luciferase reporter gene controlled by multiple STAT3-binding sites (m67-luciferase) in 293T cells that were transfected with the S3C or mS3C expression plasmids or control vector together with an EF-Renilla reporter gene to serve as an internal control for transfection efficiency.

FIG. 3B is a series of photographs of petri dishes showing the results of a colony formation assay of cell lines infected with retroviruses encoding a puromycin resistance gene together with S3C, mS3C, or the control retrovirus. S3C, but not mS3C, specifically suppressed colony formation in glioma cell lines (U87 and LN229) but not in NIH3T3 cells.

FIG. 3C (left panel) is a series of immunoblots showing the levels of STAT3, Flag, STAT1, and Akt in U87 cells stably infected with the control retrovirus containing an IRES-GFP cassette or retroviruses that also encoded FLAG-tagged S3C or mS3C.

FIG. 3C (middle panel) is a line graph showing a cell growth curve of stably infected U87 cells. S3C-expressing U87 cells proliferate significantly slower than mS3C-expressing U87 cells or those infected with control virus (n=3; ANOVA, p<0.05).

FIG. 3C (right panel) is a bar graph showing BrdU incorporation in U87 stable cell lines in culture measured as a percentage of the total number of cells. Incorporation of BrdU is significantly reduced in U87-S3C cells compared to both vector and mS3C (n=4; ANOVA, p<0.05).

FIG. 3D (left panel) is a series of photographs of tumors showing that activated STAT3 reduced glioma growth in vivo. PTEN−/− astrocytes were stably infected with the control retrovirus or the virus encoding S3C and were injected subcutaneously into SCID mice. Three weeks after injection, tumors were removed and H&E staining confirmed the presence of tumor cells. Tumors from S3C-expressing PTEN−/− astrocytes were smaller than those formed from control. Calibration bar equals 1 mm.

FIG. 3D (right panel) is a graph showing the weight in grams of excised tumors. S3C-derived tumors weighed significantly less than those formed from control cells. (n=5; t-test, p<0.05).

FIG. 3E is a series of immunoblots showing STAT3 and actin levels in PTEN flox/flox astrocytes infected with either control lentivirus or lentivirus encoding one of two small interfering hairpin RNAs directed at STAT3 and selected with puromycin. Both stat3i1 and stat3i2 hairpin RNAs induced knockdown of endogenous STAT3. Actin indicates equal protein loading.

FIG. 3F is a bar graph quantifying the invasion potential of STAT3-deficient astrocytes through a matrigel substrate. PTEN flox/flox astrocytes that were infected with the control or stat3 RNAi lentiviruses were seeded onto the top of an 8 μm pore size insert coated with matrigel. Significantly more cells infected with either stat3i1 or stat3i2 invaded the matrigel substrate than vector-infected cells (average of 2 representative experiments, performed in duplicate or quadruplicate).

FIG. 3G is a bar graph showing the invasion activity of U87 glioma cells that were infected with the control or S3Cencoding retrovirus were seeded onto the top of an 8 μm pore size insert coated with matrigel. Significantly more U87-vector glioma cells invaded the matrigel substrate than U87-S3C glioma cells. (n=3; paired t-test, p<0.05) Equivalent numbers of NIH3T3 cells failed to invade the matrigel.

FIG. 3H is a series of photographs showing Phalloidin red staining of actin stress fibers of stable U87 cell lines plated onto coverslips coated with either polyomithine (PO) or polyomithine together with myelin (20 μg/ml). U87-S3C glioma cells failed to spread on myelin (middle bottom panel), compared to a spread appearance on a polyomithine control substrate (middle top panel). U87-vector and U87-mS3C glioma cells spread and formed stress fibers on myelin (left and right bottom panels).

FIG. 3I is a bar graph quantifying cell spreading of U87 stable cell lines on myelin. Significantly fewer U87-S3C glioma cells spread on myelin compared to U87-vector glioma cells (n=3; ANOVA,p<0.0001). Cells were counted in a blinded manner in three independent experiments, and the percent spreading was determined by calculating the number of spread cells over the total number of cells.

FIG. 4A is a diagrammatic representation of the top 50 genes in three independent microarray analyses of control or S3C-expressing U87 cells, ranked according to fold change, that were repressed or induced upon S3C expression. Genes are listed in Tables 1 and 2.

FIG. 4B is a list of the top 10 repressed (left) or induced (right) genes upon S3C expression. The fold change is indicated, and GenBank numbers are in parenthesis.

FIG. 4C (left panel) is a series of photographs showing a northern analyis of control vector-infected U87 cells or those expressing S3C using an IL-8 probe.

FIG. 4C (middle panel) is a series of immunoblots showing IL-8 and actin levels in control U87 glioma cells or those expressing S3C or mS3C with the IL-8 antibody. Asterisk indicates a nonspecific band.

FIG. 4C (right panel) is a graph representing the results of a sandwich ELISA chemiluminescent analysis of medium from the control U87 glioma cells or those expressing S3C using two antibodies specific for human IL-8 (R&D Systems). Shown are mean±SEM IL-8 concentration (pg IL-8/mg cell protein; n=3; paired t-test, p<0.01).

FIG. 4D is a bar graph showing that STAT3 represses the IL-8 promoter in astrocytes as a measure of luciferase activity. PTEN−/− astrocytes were transfected with increasing amounts of an expression plasmid encoding S3C or a control plasmid together with an IL-8 promoter-controlled luciferase reporter gene and a Renilla expression plasmid and subjected to dual luciferase assay 48 hours after transfection. Activated STAT3 significantly reduced IL-8 promoter activity (n=3; ANOVA, p<0.001).

FIG. 4E is a photograph of an electrophoretic gel showing the results of a chromatin immunoprecipitation analysis at the endogenous IL-8 promoter in S3C-expressing U87 astrocytoma cells using 2 different anti-STAT3 antibodies (m, mouse; r, rabbit). Mouse and rabbit anti-HA antibodies were used as controls. Negative controls for the PCR reaction were performed with primers for the E-cadherin gene. Thus, STAT3 directly binds to the endogenous IL-8 gene.

FIG. 4F is a series of bar graphs showing that endogenous STAT3 suppresses the IL-8 promoter in astrocytes. PTEN flox/flox astrocytes stably infected with either control lentivirus or the stat3 RNAi lentiviruses were transfected with the IL-8 promoter-luciferase reporter gene and a Renilla expression plasmid and subjected to a dual luciferase assay. IL-8 promoter activity is greater in astrocytes expressing stat3i1 (n=3; paired t-test, p<0.05) or stat3i2 (n=6; paired t-test, p=0.08)

FIG. 4G is a bar graph showing that PTEN loss relieves repression of the IL-8 promoter. PTEN flox/flox or PTEN−/−astrocytes were transfected with the IL-8 promoter-luciferase reporter gene and a Renilla expression plasmid and subjected to a dual luciferase assay. IL-8 promoter activity is significantly less in PTEN flox/flox astrocytes (n=3; paired t-test, p<0.0001).

FIG. 4H (left panel) is an immunoblot showing IL-8 and actin levels in U87 cells infected with IL-8 RNAi encoding lentiviruses (IL-8i1 or IL-8i2) or an empty vector and selected with puromycin with the IL-8 or actin antibody. The asterisk indicates a non-specific band.

FIG. 4H (middle panel) is a series of bar graphs showing that IL-8 knockdown suppresses glioma cell proliferation (n=3; ANOVA, p<0.001).

FIG. 4H (right panel) is a bar graph showing the relative invasion activity of U87 glioma cells that were infected with the control lentivirus or IL-8 RNAi lentiviruses. Cells were seeded on top of an 8 μm pore size insert coated with matrigel. Significantly more U87-vector glioma cells invaded the matrigel substrate than U87-IL-8i1 or U87-IL-8i2 glioma cells (ANOVA, p<0.01).

FIG. 4I is a graph quantifying the level of normalized cell spreading. IL-8 addition rescues STAT3 suppression of glioma cell spreading on myelin. Quantification of cell spreading shows that in the presence of IL-8 significantly more U87-S3C cells spread on myelin compared to untreated U87-S3C cells plated on myelin (n=3; ANOVA, p<0.0005).

FIG. 5A is a series of immunoblots showing the level of PTEN, LIFRβ, PYS3, IL-8, and GAPDH in human glioblastomas (GBM) samples.

FIG. 5B is a bar graph showing the incidence of low PTEN, low LIFRβ, low PYS3 and high IL-8 expression within the 25 human GBM samples analyzed in FIG. 5A.

FIG. 5C is a bar graph showing the incidence of low LIFRβ, low PYS3 and high IL-8 expression within either the low PTEN or high PTEN expressing human GBMs. There were significantly more low LIFRβ, low PYS3 and high IL-8 expressing tumors in the group of GBMs that expressed low or no PTEN.

### DETAILED DESCRIPTION OF THE INVENTION

PTEN is a tumor suppressor gene located on chromosome 10. Mutation leads to an increased risk of cancer. As tumours progress to more advanced stages, they tend to acquire an increasing number of genetic alterations. One common alteration seen in a range of different advanced cancers is mutation of the PTEN gene, a gene which is linked with cell regulation and apoptosis (programmed cell death). Mutations in the PTEN gene are documented in cancers of the breast, prostate, endometrium, ovary, colon, melanoma, glioblastoma and lymphoma. A PTEN deficiency, e.g., loss of just one or both copies of the PTEN gene, is enough to interrupt cell signalling and begin the process of uncontrolled cell growth. Cancers that are PTEN-deficient include glioblastoma, breast, prostate, melanoma, endometrial, and kidney tumors.

The present invention is based on the discovery that STAT3 exerts tumor suppressive effects in glioblastomas and that IL8 promotes glioblastoma tumor proliferation and invasiveness. STAT3 exerts its tumor suppressive properties, at least in part, by repressing the expression of IL-8. IL-8 is a chemokine that regulates neutrophil activation and chemoattraction. In addition, IL-8 has been implicated in the promotion of angiogenesis in a variety of tumors including glioma. Consistent with a role for IL-8 in glioma progression, although IL-8 expression is very low in normal brain, IL-8 is upregulated in gliomas and in particular in high grade astrocytomas and glioblastomas.

The PTEN-STAT3 signaling pathway also plays a role in the incidence and progression of tumors in tissues/organs outside the brain. Although the PTEN-regulated STAT3 tumor suppressive pathway described herein was first identified in glial cells and found to contribute to the pathogenesis of brain tumors, the findings are relevant to a whole host of other types of tumors that develop outside the brain. For example, loss of PTEN is an important causative factor in breast and prostate cancer. Therefore, the characterization of STAT3's tumor suppressive properties in glial cells is applicable to prostate and breast cancer as well as other cancers in which PTEN loss is an important factor. Therefore, in the class of tumors characterized by PTEN decrease or loss, small molecule activators of STAT3 or small molecule inhibitors of the STAT3-repressed target gene IL8 confer clinical benefit and are useful as a treatment modality alone or in conjunction with other therapeutic interventions such as surgery, radiation, or chemotherapy.

Accordingly, the methods and compositions provided herein are useful for treating and preventing brain tumors (e.g., gliomas) by administering to a subject in need thereof an agent that reduces the expression or activity of IL-8 or the IL-8 receptor (IL-8R) in neurons such as glioma cells. One exemplary agent is the small molecule repertaxin. Other small molecule IL-8 receptor antagonists include SB455821 (a synthetic, non-peptide CXCR2 antagonist (Glaxo SmithKline Beecham); Immunobiology, 2004, 209:225-33)), SB265610 (Glaxo SmithKline Beecham; Benson et al., 2000, 151:196-197), as well as SCH 527123 (2-hydroxy-N,N-dimethyl-3-{2-[[(R)-1-(5-methylfuran-2-yl)propyl]amino]-3,4-dioxocyclobut-1-enylamino} benzamide (SCH 527123), an orally bioavailable CXCR2/CXCR1 receptor antagonist (Schering Plough)).

Since STAT3 acts as a repressor of IL-8 expression, such reduction may involve the induction in the expression or activity of STAT3. If desired, a second therapeutic regimen may also be used in the present invention including, for example, surgery (tumor resection), chemotherapy, or radiotherapy.

The experiments described herein were performed using the following Materials and Methods.

Cell Culture

PTEN flox/flox and PTEN −/− astrocytes and MEFs were cultured from ink4/Arf−/− mice in which the PTEN gene contained loxP sites flanking exon five.

Plasmids

The RcCMV/mS3C, MSCV/mS3C-GFP, and pBABE/mS3C constructs were made by site directed mutagenesis of RcCMV/S3C, MSCV/S3C-GFP, and pBABE/S3C 21 using the QuikChange Site-Directed Mutagenesis Kit (Stratagene). MSCV/STAT3-GFP and MSCV/S3DGFP were generated by subcloning STAT3 and S3D (dominant-negative STAT3) from pEFBOS constructs into MSCV-GFP. Genes of interest were subcloned into MSCV-GFP to be expressed under the control of the MSCV promoter and upstream of an IRES-GFP cassette. All plasmids were sequenced.

Virus Production and Infection

Recombinant retroviruses were made by transfecting 293T cells with pMD.MLV gag.pol, pHDM.G (VSVG pseudotype), and the transfer plasmid (i.e. pBABE). Cells were infected with equal amounts of retroviruses in the presence of 8 μg/ml polybrene (Sigma). A population of infected cells for each construct was collected by sorting cells for GFP expression. PTEN−/− astrocytes were infected with pBABE or pBABE/S3C retroviruses and infected cells were selected with puromycin (4 μg/ml). PTEN flox/flox astrocytes were infected with MyrAkt and infected cells were selected with 250 μg/ml hygromycin (Invitrogen). Cloning of recombinant lentiviruses coding for a short hairpin RNA directed against STAT3 or human IL-8 was carried out using the pLL3.7 vector. The following complementary oligonucleotides were inserted into pLL3.7: stat3i1 fw, 5′-TGG AGT GTG CTA CCT CCC CGT TCA AGA GAC GGG GAG GTA GCA CAC TCC TTT TTG GAA AC-3′; stat3i1 rev, 5′-TCG AGT TTC CAA AAA GGA GTG TGC TAC CTC CCC GTC TCT TGA ACG GGG AGG TAG CAC ACT CCA-3′.stat3i2 fw, 5′-TGG GCA GTT TGA GTC GCT CAT TCA AGA GAT GAG CGA CTC AAA CTG CCC TTT TTG GAA AC-3′; stat3i2 rev, 5′-TCG AGT TTC CAA AAA GGG CAG TTT GAG TCG CTC ATC TCT TGA ATG AGC GAC TCA AAC TGC CCA-3′. IL-8i1 fw, 5′-TGA ACT TAG ATG TCA GTG CAT TCA AGA GAT GCA CTG ACA TCT AAG TTC TTT TTG GAA AC-3′; IL-8i1 rev, 5′-TCG AGT TTC CAA AAA GAA CTT AGA TGT CAG TGC ATC TCT TGA ATG CAC TGA CAT CTA AGT TCA-3′. IL-8i2 fw, 5′-TGA ACT GAG AGT GAT TGA GAG TTC AAG AGA CTC TCA ATC ACT CTC AGT TCT TTT TGG AAA C-3′; IL-8i2 rev, 5′-TCG AGT TTC CAA AAA GAA CTG AGA GTG ATT GAG AGT CTC TTGAAC TCT CAA TCA CTC TCA GTT CA-3′. Hairpin structures containing the stem.sequences (underlined) and the loops (bold italica) are indicated. Lentiviruses were generated by co-transfecting pLL3.7 and packaging vectors (VSVG, RSV-REV and pMDL g/p RRE) into 293T cells. Infected cells were selected with puromycin.

Mouse Injections

1×10⁶ cells were resuspended in serum-free media and injected bilaterally and subcutaneously into 4-6 week old SCID mice. Approximately three weeks after injection, the mice were sacrificed and the tumors removed and weighed.

Microarray Analysis

RNA was extracted from U87 stable cell lines using Trizol, followed by an additional purification step using an RNAeasy kit (Qiagen) according to the manufacturer's instructions. Biotinylated cRNAs from each cell line were generated from 15 μg of total RNA and hybridized to the Affymetrix U133A chips. Each cell line was used in three separate experiments. Gene expression data was analyzed using Vector Xpression software (InforMax Inc.). Raw expression values were normalized by linear scaling so that the mean array intensity was identical for all scans. Intensity thresholds were set a min=20 and max=16,000 units, resulting in 12,284 probe sets for subsequent analysis. These remaining 12,284 probe sets were then subjected to the t-test using Vector Xpression for the identification of differentially expressed transcripts. Fold change expression data was diagrammatically represented using GeneCluster software (wwwgenome.wi.mit.edu/cancer/software/software.html).

Matrigel Invasion assays

Matrigel pre-coated invasion chambers (Becton Dickinson) with a 8 μm pore size membrane were utilized according to the manufacturer's instructions. 2.5×10⁴ cells in 500 μl of serum-free DMEM were added to each of the inserts and incubated at 37° C. for 22 hours. Cells on the lower surface of the membrane which had migrated through the matrigel were fixed, stained with crystal violet, and counted.

Chromatin Immunoprecipitation

Chromatin immunoprecipitation analyses were done as described. Following immunoprecipitation, a PCR reaction was used to amplify the IL-8 promoter with the following primers: 1ₛₜ PCR reaction, IL8 fw, 5′-TCT CAC TCC ATC CCT TTT GC-3′, IL8 rev, 5′-AGT GGC AGG TGT TAG AAC AAG A-3′; nested PCR reaction, nested IL8 fw, 5′-CTC CAT CCC TTT TGC TAG TGA-3′, nested IL8 rev, 5′-ACA GAT GCT ATC ATG ATG GTG AA-3′.

Cell Spreading Assays

1×10⁴ cells were seeded onto glass coverslips pre-coated with polyornithine (15 mg/ml) and additionally coated with PBS or 20 μg/ml myelin. Where indicated recombinant human IL-8 (R&D) was added at a concentration of 50 ng/ml. Cells were incubated at 37° C. for 1 hour to attach and then fixed with 4% paraformaldehyde for 20 minutes at RT. Coverslips were stained with phallotoxin for 20 minutes according to the manufacturer's directions (Molecular probes). Nuclei were counterstained with 0.2 μg/ml Hoechst (Sigma). Spread versus unspread cells were counted blind, and a percentage of spread cells over the total number of cells calculated. Pictures were taken at 60× using a Nikon inverted microscope.

Cell Signaling in Gliomas

Few tumors carry worse prognosis than the malignant gliomas, the most common primary tumors of the brain. The failed record of clinical trials in the treatment of these tumors may reflect the unique biology of glioma cells, including their selective ability to infiltrate normal brain tissue and migrate along white matter tracts. Mutations in the phosphatase PTEN comprise a common genetic abnormality that promotes glioma progression.

The tumor suppressor PTEN functions in several tissues including brain, where PTEN loss facilitates glioma progression. The role of PTEN on the malignant behavior of glioma cells including their proliferation and propensity for invasiveness was investigated. Results show that PTEN deficiency triggered specific inhibition of the gliogenesis-promoting LIFRβ-STAT3 signaling pathway in astrocytes but not in non-neural cells. Strikingly, in contrast to STAT3's oncogenic role elsewhere, reactivation of STAT3 in PTEN-deficient astrocytes or glioma cells inhibited cell proliferation and glioma growth in vivo, and suppressed glioma cell invasiveness and spreading on myelin. The chemokine IL-8 was identified as a direct target gene of STAT3 in glioma cells, whose repression mediated STAT3-inhibition of glioma cell proliferation, invasiveness, and spreading on myelin. In human glioblastoma specimens, PTEN loss was found to be associated with low LIFRβ, low phosphorylated STAT3, and high IL-8 expression. Together, the findings indicate that suppression of the LIFRβ-STAT3 signaling pathway plays a key role in PTEN loss-induced glioma cell proliferation and invasiveness. Thus, deregulation of developmental signaling pathways that control gliogenesis are intimately linked to the unique biological behaviors of gliomas.

Therapeutic Agents

An inhibitor of IL-8 or IL-8R is any agent having the ability to reduce the expression or the activity of IL-8 or IL-8R in a cell concomitant with a reduction in IL-8R signaling. Thus, reducing IL-8R activity may be associated with an increase in STAT3 expression or activity. The control cell is a cell that has not been treated with the IL-8 or IL-8R inhibitor or the STAT3 activator. IL-8 (or IL-8R) expression or activity is determined by any standard method in the art, including those described herein. IL-8 or IL-8R inhibitors include polypeptides, polynucleotides, small molecule antagonists, or siRNA.

An IL-8 or IL-8R inhibitor polypeptide includes, for example, an antibody or fragment thereof that reduces IL-8 or IL-8R expression or signaling. Exemplary antibodies include anti-IL-8R antibodies and anti-IL-8 antibodies.

Alternatively, the IL-8 or IL-8R inhibitor is a dominant negative protein or a nucleic acid encoding a dominant negative protein that interferes with the biological activity of IL-8 or IL-8R. A dominant negative protein is any amino acid molecule having a sequence that has at least 50%, 70%, 80%, 90%, 95%, or even 99% sequence identity to at least 10, 20, 35, 50, 100, or more than 150 amino acids of the wild type protein to which the dominant negative protein corresponds. For example, a dominant-negative IL-8R has mutation such that it no longer activates downstream pathways. Alternatively, the IL-8 or IL-8R inhibitor may be a dominant active STAT3 protein or a nucleic acid encoding a dominant active STAT3 protein having an increase in biological activity (e.g., at least 10%, 20%, 30%, 40%, 50%, 60%, 70%, 80%, 90%, 100% or more) relative to the naturally-occurring STAT3 protein.

The dominant negative or dominant active protein may be administered as an expression vector. The expression vector may be a non-viral vector or a viral vector (e.g., recombinant retrovirus, recombinant lentivirus, recombinant adeno-associated virus, or a recombinant adenoviral vector). Alternatively, the dominant negative protein may be directly administered as a recombinant protein systemically or to the infected area using, for example, microinjection techniques.

The IL-8 or IL-8R inhibitor is an antisense molecule, an RNA interference (siRNA) molecule, or a small molecule antagonist that targets IL-8 or IL-8R expression or activity. By the term “siRNA” is meant a double stranded RNA molecule which prevents translation of a target mRNA. Standard techniques of introducing siRNA into a cell are used, including those in which DNA is a template from which an siRNA RNA is transcribed. The siRNA includes a sense IL-8 or IL-8R nucleic acid sequence, an anti-sense IL-8 or IL-8R nucleic acid sequence or both. Optionally, the siRNA is constructed such that a single transcript has both the sense and complementary antisense sequences from the target gene, e.g., a hairpin. Binding of the siRNA to an IL-8 or IL-8R transcript in the target cell results in a reduction in IL-8 or IL-8R production by the cell. The length of the oligonucleotide is at least 10 nucleotides and may be as long as the naturally-occurring IL-8 or IL-8R transcript. Preferably, the oligonucleotide is 19-25 nucleotides in length. Most preferably, the oligonucleotide is less than 75, 50 , 25 nucleotides in length.

Small molecules includes, but are not limited to, peptides, peptidomimetics (e.g., peptoids), amino acids, amino acid analogs, polynucleotides, polynucleotide analogs, nucleotides, nucleotide analogs, organic and inorganic compounds (including heterorganic and organomettallic compounds) having a molecular weight less than about 5,000 grams per mole, organic or inorganic compounds having a molecular weight less than about 2,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Useful small molecules may reduce IL-8 expression or activity, or IL-8R activity) by increasing STAT3 expression or activity. Exemplary small molecules are CXCR antagonists, such as CXCR1 antagonists (e.g., repertaxin) and CXCR2 antagonists (e.g.: SB-332235-Z). Such inhibitors are described, for example, in Bertini et al., Proc. Natl. Acad. Sci. USA, 101:11791-6, (2004), White et al., J. Biol. Chem. 273: 10095-8 (1998), all of which are hereby incorporated by reference.

The preferred dose of the IL-8R or IL-8 inhibitor is a biologically active dose. A biologically active dose is a dose that will reduce the proliferation and invasive abilities of glioma cells. Desirably, the IL-8 or IL-8R inhibitor has the ability to reduce the expression or activity of IL-8R in neuronal cells (e.g., glioma cells) by at least 5%, 10%, 20%, 30%, 40%, 50%, 60%, 70%, 80%, 90%, or 100% below untreated control levels. The levels or activity of IL-8R or IL-8 in cells is measured by any method known in the art, including, for example, Western blot analysis, immunohistochemistry, ELISA, and Northern Blot analysis. Alternatively, the biological activity of IL-8 or IL-8R is measured by assessing the expression or activity of any of the molecules involved in IL-8R signaling. The biological activity of IL-8 is determined according to its ability to increase cell proliferation or its ability to increase tumor cell invasiveness. Preferably, the agent that reduces the expression or activity of IL-8 or IL-8R can reduce glioma cell proliferation, invasiveness, or both by at least 5%, 10%, 20%, 30%, 40%, 50%, 60%, 70%, 80%, 90%, or 100% below untreated control levels. The agent of the present invention is therefore any agent having any one or more of these activities.

Optionally, the subject is administered one or more additional therapeutic regiments. Additional therapeutic regimens include, for example, chemotherapeutic agents (e.g., asparaginase, bleomycin, busulfan carmustine (commonly referred to as BCNU), chlorambucil, cladribine (commonly referred to as 2-CdA), irinotecan (CPT-11), cyclophosphamide, cytarabine (commonly referred to as Ara-C), dacarbazine, daunorubicin, dexamethasone, doxorubicin (commonly referred to as Adriamycin), etoposide, fludarabine, 5-fluorouracil (commonly referred to as 5FU), hydroxyurea, idarubicin, ifosfamide, interferon-α (native or recombinant), levamisole, lomustine (commonly referred to as CCNU), mechlorethamine (commonly referred to as nitrogen mustard), melphalan, mercaptopurine, methotrexate, mitomycin, mitoxantrone, paclitaxel, pentostatin, prednisone, procarbazine, tamoxifen, taxol-related compounds, 6-thiogaunine, topotecan, vinblastine, and vincristine), gabapentin, toiramate, lamotrigine, tiagabine, radiotherapy, and surgery. The additional therapeutic regimens may be administered prior to, concomitantly, or subsequent to administration of the IL-8 or IL-8R inhibitor. For example, the IL-8 or IL-8R inhibitor and the additional agent are administered in separate formulations within at least 1, 2, 4, 6, 10, 12, 18, or more than 24 hours apart. Optionally, the additional agent is formulated together with the IL-8 or IL-8R inhibitor. When the additional agent is present in a different composition, different routes of administration may be used. The agent is administered at doses known to be effective for such agent for treating, reducing, or preventing the growth or invasiveness of a tumor.

Concentrations of the IL-8 or IL-8R inhibitor and the additional agent depends upon different factors, including means of administration, target site, physiological state of the mammal, and other medication administered. Thus treatment dosages may be titrated to optimize safety and efficacy and is within the skill of an artisan. Determination of the proper dosage and administration regime for a particular situation is within the skill of the art.

IL-8 or IL-8R inhibitors are administered in an amount sufficient to reduce the proliferation, invasive ability (e.g., migration), or both of tumor cells. Such reduction includes the alleviation of one or more of symptoms associated with brain tumors. Administration of the IL-8 or IL-8R inhibitor reduces the brain tumor or alleviates one or more symptoms associated with the brain tumor by at least 10%, 20%, 30%, 40%, 50%, 60%, 70%, 80%, 90%, or 100% as compared to an untreated subject.

Treatment is efficacious if the treatment leads to clinical benefit such as, a reduction of the symptoms of a brain tumor in the subject. When treatment is applied prophylactically, “efficacious” means that the treatment retards or prevents a brain tumor from forming. Efficacy may be determined using any known method for diagnosing or treating the tumor.

Therapeutic Administration

The invention includes administering to a subject a composition that includes a compound that reduces IL-8R expression or activity (referred to herein as an “IL-8R inhibitor” or “therapeutic compound”). As described herein, this inhibitor may also increase STAT3 expression or activity.

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-neoplastic agents or therapeutic agents for treating, preventing or alleviating a symptom of a brain tumor. A therapeutic regimen is carried out by identifying a mammal, e.g., a human patient suffering from (or at risk of developing) a brain tumor, using standard methods.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The compound is administered prophylactically, or after the detection of a brain tumor. The compound is optionally formulated as a component of a cocktail of therapeutic drugs to treat the brain tumor. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the IL-8R inhibitor is formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, suppository, paste, spray, patch, cream, gel, resorbable sponge, or foam. Such formulations are produced using methods well known in the art.

Where the therapeutic compound is a nucleic acid encoding a protein, the Therapeutic nucleic acid is administered in vivo to promote expression of its encoded protein, by constructing it as part of an-appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. A nucleic acid therapeutic is introduced intracellularly and incorporated within host cell DNA or remain episomal.

For local administration of DNA, standard gene therapy vectors used. Such vectors include viral vectors, including those derived from replication-defective hepatitis viruses (e.g., HBV and HCV), retroviruses (see, e.g., WO 89/07136; Rosenberg et al., 1990, N. Eng. J. Med. 323(9):570-578), adenovirus (see, e.g., Morsey et al., 1993, J. Cell. Biochem., Supp. 17E,), adeno-associated virus (Kotin et al., 1990, Proc. Natl. Acad. Sci. USA 87:2211-2215,), replication defective herpes simplex viruses (HSV; Lu et al., 1992, Abstract, page 66, Abstracts of the Meeting on Gene Therapy, Sept. 22-26, Cold Spring Harbor Laboratory, Cold Spring Harbor, New York), and any modified versions of these vectors. The invention may utilize any other delivery system which accomplishes in vivo transfer of nucleic acids into eucaryotic cells. For example, the nucleic acids may be packaged into liposomes, e.g., cationic liposomes (Lipofectin), receptor-mediated delivery systems, non-viral nucleic acid-based vectors, erythrocyte ghosts, or microspheres (e.g., microparticles; see, e.g., U.S. Pat. No. 4,789,734; U.S. Pat. No. 4,925,673; U.S. Pat. No. 3,625,214; Gregoriadis, 1979, Drug Carriers in Biology and Medicine, pp. 287-341 (Academic Press,). Naked DNA may also be administered.

DNA for gene therapy can be administered to patients parenterally, e.g., intravenously, subcutaneously, intramuscularly, and intraperitoneally. DNA or an inducing agent is administered in a pharmaceutically acceptable carrier, i.e., a biologically compatible vehicle which is suitable for administration to an animal e.g., physiological saline. A therapeutically effective amount is an amount which is capable of producing a medically desirable result, e.g., a decrease of an IL-8R gene product in a treated animal. Such an amount can be determined by one of ordinary skill in the art. As is well known in the medical arts, dosage for any given patient depends upon many factors, including the patient's size, body surface area, age, the particular compound to be administered, sex, time and route of administration, general health, and other drugs being administered concurrently. Dosages may vary, but a preferred dosage for intravenous administration of DNA is approximately 10⁶ to 10²² copies of the DNA molecule. Typically, plasmids are administered to a mammal in an amount of about 1 nanogram to about 5000 micrograms of DNA. Desirably, compositions contain about 5 nanograms to 1000 micrograms of DNA, 10 nanograms to 800 micrograms of DNA, 0.1 micrograms to 500 micrograms of DNA, 1 microgram to 350 micrograms of DNA, 25 micrograms to 250 micrograms of DNA, or 100 micrograms to 200 micrograms of DNA. Alternatively, administration of recombinant adenoviral vectors encoding the IL-8R inhibitor into a mammal may be administered at a concentration of at least 10⁵, 10⁶, 10⁷, 10⁸, 10⁹, 10¹⁰, or 10¹¹ plaque forming unit (pfu).

IL-8R gene products are administered to the patient intravenously in a pharmaceutically acceptable carrier such as physiological saline. Standard methods for intracellular delivery of peptides can be used, e.g. packaged in liposomes. Such methods are well known to those of ordinary skill in the art. It is expected that an intravenous dosage of approximately 1 to 100 moles of the polypeptide of the invention would be administered per kg of body weight per day. The compositions of the invention are useful for parenteral administration, such as intravenous, subcutaneous, intramuscular, and intraperitoneal.

IL-8 or IL-8R inhibitors are effective upon direct contact of the compound with the affected tissue or may alternatively be administered systemically (e.g., intravenously, rectally or orally). The IL-8 or IL-8R inhibitor may be administered intravenously or intrathecally (i.e., by direct infusion into the cerebrospinal fluid). For local administration, a compound-impregnated wafer or resorbable sponge is placed in direct contact with CNS tissue. The compound or mixture of compounds is slowly released in vivo by diffusion of the drug from the wafer and erosion of the polymer matrix. Alternatively, the compound is infused into the brain or cerebrospinal fluid using standard methods. For example, a burr hole ring with a catheter for use as an injection port is positioned to engage the skull at a burr hole drilled into the skull. A fluid reservoir connected to the catheter is accessed by a needle or stylet inserted through a septum positioned over the top of the burr hole ring. A catheter assembly (described, for example, in U.S. Pat. No. 5,954,687) provides a fluid flow path suitable for the transfer of fluids to or from selected location at, near or within the brain to allow administration of the drug over a period of time.

One in the art will understand that the patients treated according to the invention may have been subjected to the tests to diagnose a subject as having a brain tumor or may have been identified, without examination, as one at high risk due to the presence of one or more risk factors (e.g., genetic predisposition). Reduction of brain tumor symptoms or damage may also include, but are not limited to, alleviation of symptoms (e.g., headaches, nausea, skin rash), diminishment of extent of disease, stabilized (i.e., not worsening) state of disease, delay or slowing of disease progression, and amelioration or palliation of the disease state. Treatment may occur at home with close supervision by the health care provider, or may occur in a health care facility.

Screening Assays

The present invention provides screening methods to identify compounds that can inhibit the expression or activity of IL-8 or IL-8R. Useful compounds include any agent that inhibits the biological activity or reduces the cellular level of IL-8 or IL-8R. For example, useful compounds are identified by detecting an attenuation of the expression or activity of any of the molecules involved in IL-8R signaling. The screening assays may also identify agents that increase the expression or activity level of STAT3.

A number of methods are available for carrying out such screening assays. According to one approach, candidate compounds are added at varying concentrations to the culture medium of cells expressing IL-8R. Gene expression of IL-8R is then measured, for example, by standard Northern blot analysis, using any appropriate fragment prepared from the nucleic acid molecule of IL-8R as a hybridization probe or by real time PCR with appropriate primers. The level of gene expression in the presence of the candidate compound is compared to the level measured in a control culture medium lacking the candidate molecule. If desired, the effect of candidate compounds may, in the alternative, be measured at the level of IL-8R polypeptide using the same general approach and standard immunological techniques, such as Western blotting or immunoprecipitation with an antibody specific to IL-8R for example. For example, immunoassays may be used to detect or monitor the level of IL-8R. Polyclonal or monoclonal antibodies which are capable of binding to IL-8R may be used in any standard immunoassay format (e.g., ELISA or RIA assay) to measure the levels of IL-8R. IL-8R can also be measured using mass spectroscopy, high performance liquid chromatography, spectrophotometric or fluorometric techniques, or combinations thereof.

As a specific example, mammalian cells (e.g., rodent cells) that express a nucleic acid encoding IL-8R are cultured in the presence of a candidate compound (e.g., a peptide, polypeptide, synthetic organic molecule, naturally occurring organic molecule, nucleic acid molecule, or component thereof). Cells may either endogenously express IL-8R or may alternatively be genetically engineered by any standard technique known in the art (e.g., transfection and viral infection) to overexpress IL-8R. The expression level of IL-8R is measured in these cells by means of Western blot analysis and subsequently compared to the level of expression of the same protein in control cells that have not been contacted by the candidate compound. A compound which promotes a decrease in the level of IL-8R activity as a result of reducing its synthesis or biological activity is considered useful in the invention.

Alternatively, the screening methods of the invention may be used to identify candidate compounds that decrease the biological activity of IL-8R by increasing the biological activity or expression of STAT3 by at least 10%, 20%, 30%, 40%, 50%, 60%, 70%, 80%, 90%, 95%, or 100% relative to an untreated control. As an example, a candidate compound may be tested for its ability to increase STAT3 activity in cells that naturally express STAT3, after transfection with cDNA for STAT3, or in cell-free solutions containing STAT3, as described further below. The effect of a candidate compound on the binding or activation of STAT3 can be tested by radioactive and non-radiaoctive binding assays, competition assays, and receptor signaling assays.

Given its ability to decrease the biological activity of IL-8 or IL-8R, such a molecule may be used, for example, as a therapeutic agent to treat, reduce, or prevent a brain tumor, or alternatively, to alleviate one or more symptoms associated with such a tumor. As a specific example, a candidate compound may be contacted with two proteins, the first protein being a polypeptide substantially identical to IL-8R and the second protein being IL-8 (i.e., a protein that binds the IL-8R polypeptide under conditions that allow binding). According to this particular screening method, the interaction between these two proteins is measured following the addition of a candidate compound. A decrease in the binding of IL-8 to IL-8R following the addition of the candidate compound (relative to such binding in the absence of the compound) identifies the candidate compound as having the ability to inhibit the interaction between the two proteins, and thereby having the ability to reduce IL-8R activity. The screening assay of the invention may be carried out, for example, in a cell-free system or using a yeast two-hybrid system. If desired, one of the proteins or the candidate compound may be immobilized on a support as described above or may have a detectable group.

Alternatively, or in addition, candidate compounds may be screened for those which specifically bind to and thereby inhibit IL-8R. The efficacy of such a candidate compound is dependent upon its ability to interact with IL-8R. Such an interaction can be readily assayed using any number of standard binding techniques and functional assays. For example, a candidate compound may be tested in vitro for interaction and binding with IL-8R and its ability to modulate tumor cell responses may be assayed by any standard assays (e.g., those described herein).

For example, a candidate compound that binds to IL-8R may be identified using a chromatography-based technique. For example, a recombinant IL-8R may be purified by standard techniques from cells engineered to express IL-8R (e.g., those described above) and may be immobilized on a column. Alternatively, the naturally-occurring IL-8R may be immobilized on a column. A solution of candidate compounds is then passed through the column, and a compound specific for IL-8R is identified on the basis of its ability to bind to IL-8R and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography).

Screening for new inhibitors and optimization of lead compounds may be assessed, for example, by assessing their ability to modulate IL-8R activity using standard techniques. In addition, these candidate compounds may be tested for their ability to function as anti-neoplastic agents (e.g., as described herein). Compounds isolated by this approach may also be used, for example, as therapeutics to treat, reduce, or prevent brain tumors, or alternatively, to alleviate one or more symptoms associated with such tumors. Compounds which are identified as binding to IL-8R with an affinity constant less than or equal to 10 mM are considered particularly useful in the invention.

Potential therapeutic agents include organic molecules, peptides, peptide mimetics, polypeptides, and antibodies that bind to a nucleic acid sequence or polypeptide that encodes IL-8R and thereby inhibit or extinguish their activity. Potential anti-microbial agents also include small molecules that bind to and occupy the binding site of such polypeptides thereby preventing binding to cellular binding molecules, such that normal biological activity is prevented. Other potential anti-neoplastic agents include antisense molecules.

This invention is based in part on the experiments described in the following examples. These examples are provided to illustrate the invention and should not be construed as limiting.

Inhibition of Neural Tumors

Gliomas are thought to arise from the malignant transformation of astrocytes and/or neural stem cells. During normal brain development, activation of the cytokine receptor leukemia inhibitory factor receptorβ (LIFRβ) and downstream STAT3 signaling pathway stimulates the differentiation of neural stem cells into astrocytes. We reasoned that glioma cell-specific pathological behaviors including their high capacity for invasiveness of CNS tumors (e.g., gliomas, ependymomas, meningiomas, pituary adenomas, acoustic Schwanoma (neuroma), medulloblastomas, brain stem gliomas, and optic nerve gliomas) might be triggered in neural stem cells or astrocytes by deregulation of signaling pathways that promote the differentiation of these cells during normal development. The role of the gliogenesis-promoting LIFRβ-STAT3 signaling pathway in glial tumorigenesis was studied and characterized.

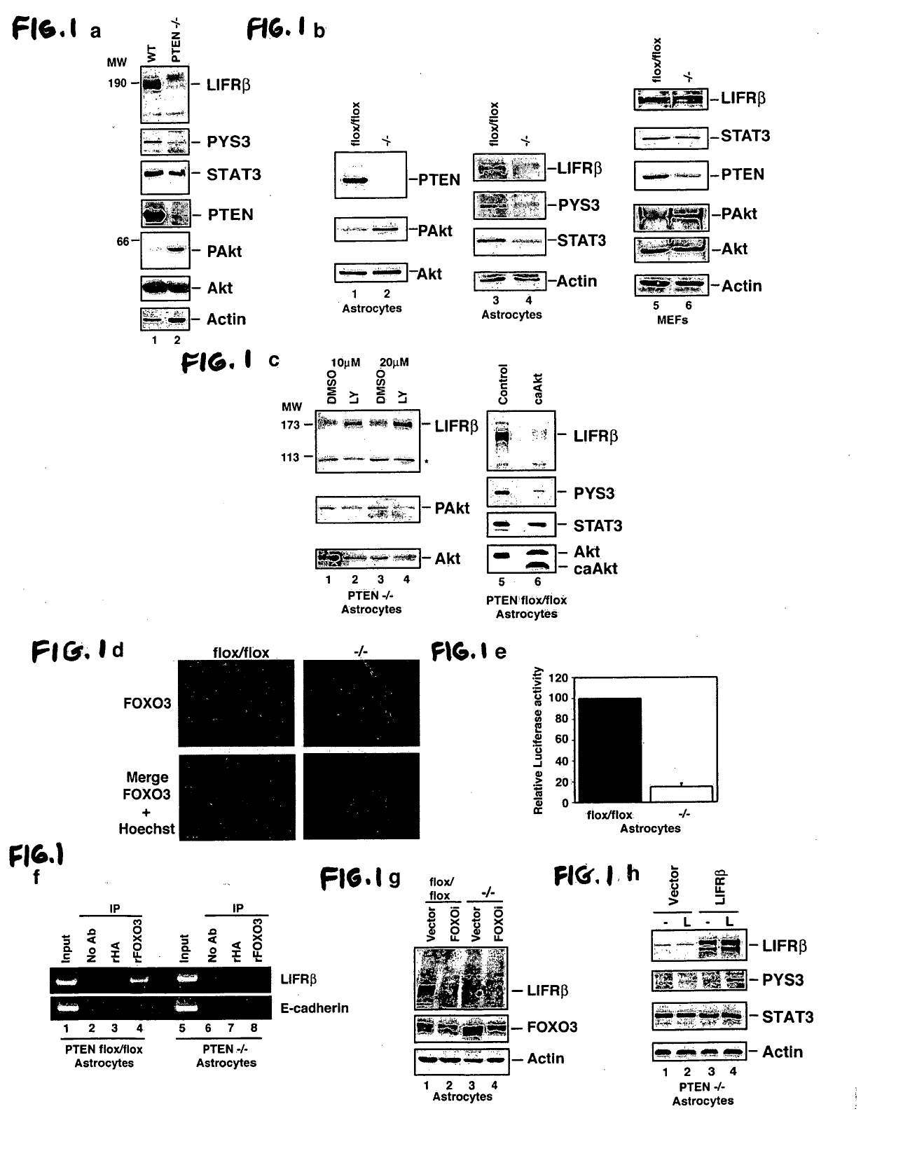

We first determined if PTEN loss impacts on the LIFRβ-STAT3 signaling pathway in glial cells. We measured the levels of LIFRβ in astrocytes in which the PTEN gene was disrupted by Cre-mediated excision of floxed PTEN alleles. As predicted, little or no PTEN protein was detected in the PTEN−/− astrocytes, and this was associated with enhanced phosphorylation of the serine/threonine kinase Akt on Serine 473 that reflects Akt activation (FIGS. 1A-1C). The amount of LIFRβ was dramatically reduced in astrocytes in which the PTEN gene was disrupted when compared to wild type astrocytes or astrocytes with homozygous floxed PTEN allele (PTEN flox/flox) (FIGS. 1A and 1B). By contrast, PTEN loss failed to inhibit LIFRβ expression in other cells including embryonic fibroblasts (FIG. 1B). These findings indicate that PTEN loss inhibits LIFRβ expression specifically in astrocytes.

We next characterized the mechanism by which PTEN deficiency suppresses LIFRβ expression. PTEN catalyzes the dephosphorylation of the lipid messenger PIP3, that is generated by the lipid kinase PI-3K. Incubation of PTEN-deficient astrocytes with the PI-3K inhibitor LY294002 restored LIFRβ expression in these cells suggesting that PTEN loss suppresses LIFRβ expression via PIP3 (FIG. 1C). Expression of a constitutive active Akt, a critical effector of PIP3₁₇, in PTEN flox/flox cells suppressed LIFRβ expression (FIG. 1C). Together, these results suggest that Akt inhibits LIFRβ expression in PTEN-deficient astrocytes.

A major substrate of Akt is the transcription factor FOXO3, whose phosphorylation inhibits FOXO-dependent transcription. Whether the LIFRβ gene might represent a direct target of FOXO3 in astrocytes and thus provide the basis for PTEN loss- and Akt-suppression of LIFRβ expression was next determined. Consistent with the idea that PTEN loss triggers the inhibition of LIFRβ gene expression, the levels of LIFRβ mRNA measured by RT-PCR analysis were dramatically reduced in PTEN-deficient astrocytes. PTEN loss was next confirmed to inhibit FOXO3 function in astrocytes. Endogenous FOXO3 was excluded from the nucleus and FOXO-dependent transcription was inhibited in astrocytes upon PTEN loss (FIGS. 1D and 1E).

The role of FOXO3 in the regulation of the LIFRβ gene was next determined. By sequence gazing, we found a conserved FOXO binding site within the promoter of the LIFRβ gene. By chromatin immunoprecipitation analyses, endogenous FOXO3 was found to occupy the endogenous LIFRβ gene in PTEN flox/flox astrocytes (FIG. 1F). However, remarkably no FOXO3 was found at the LIFRβ gene in PTENdeficient astrocytes, suggesting that FOXO3 occupancy at the LIFRβ gene correlates tightly with PTEN status and LIFRβ expression in astrocytes (FIG. 1F).

To determine the role of endogenous FOXO3 in the regulation of LIFRβ expression in astrocytes, we used a lentiviral DNA-based template method of RNA interference to knockdown FOXO3 in these cells. We found that FOXO3 knockdown in PTEN-expressing (PTEN flox/flox) astrocytes suppressed LIFRβ expression, mimicking the effect of PTEN deficiency (FIG. 1G). Taken together, our findings suggest that LIFRβ is a direct target of FOXO3 in astrocytes and support the conclusion that PTEN loss and consequent Akt activation inhibit FOXO3-dependent LIFRβ gene expression in astrocytes.

To determine the consequences of the suppression of LIFRβ expression in astrocytes, we measured the level of STAT3 that is phosphorylated at the key regulatory site of Tyr 705 in these cells. The Tyr 705 phosphorylation promotes STAT3 dimerization and translocation to the nucleus, where STAT3 regulates transcription of cytokine-responsive geneS₂₀. PTEN loss, or the expression of a constitutively active Akt, in astrocytes significantly inhibited STAT3 phosphorylation and modestly reduced STAT3 levels concomitantly with the inhibition of LIFRβ expression (FIGS. 1A-1C). Expression of exogenous LIFRβ in PTEN−/− astrocytes restored the STAT3 phosphorylation upon exposure of these cells to LIF (FIG. 1H). Together, these results suggest that PTEN deficiency suppresses the LIFRβ-STAT3 signaling pathway in astrocytes.

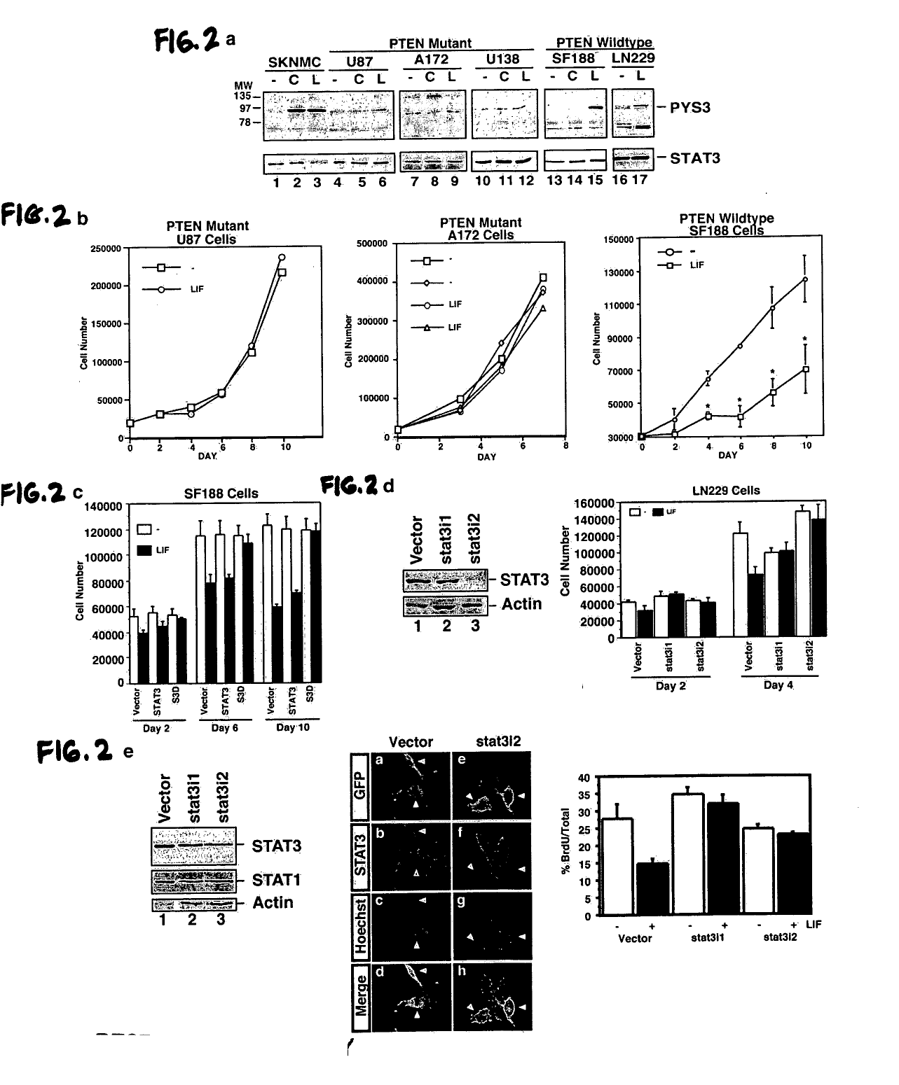

The inhibition of the STAT3 signaling pathway was also evident in human glioma cell lines that harbor PTEN mutations. While LIF induced STAT3 Tyr 705 phosphorylation in the wild type PTEN-expressing glioma cell lines SF188 and LN229, LIF failed to effectively induce the STAT3 phosphorylation in several PTEN-deficient glioma cell lines (FIG. 2A), supporting the conclusion that PTEN deficiency suppresses the STAT3 signaling pathway in glioma cells. In parallel with these results, we found that LIF had distinct effects on the proliferation of glioma cells depending on their PTEN status. While LIF inhibited the proliferation of the PTENexpressing glioma cell lines SF188 and LN229 (FIGS. 2B-2D), LIF had little effect on the proliferation of the PTEN-deficient cell lines U87 and A172 (FIG. 2B), suggesting that LIF inhibition of cell proliferation correlates with LIF-induction of endogenous STAT3 in PTEN expressing glioma cells.

To determine the role of endogenous STAT3 activation in LIF-inhibition of cell proliferation in PTEN-expressing glioma cells, we expressed in SF188 cells a dominant interfering form of STAT3 (S3D) that forms dimers with endogenous STAT3 that fail to bind to promoters of cytokine-responsive genes. We found that the expression of S3D but not wild type STAT3 significantly reduced the ability of LIF to inhibit SF188 glioma population growth (FIG. 2C). We also used a lentiviral DNA-based template method of RNA interference to knockdown STAT3 protein in the PTEN-expressing LN229 glioma cells. Two hairpin RNAs to STAT3 (stat3i1 and stat3i2) that reduced the expression of endogenous human STAT3 with increasing effectiveness were found to significantly inhibit LIF-suppression of proliferation of LN229 glioma cells (FIG. 2D). In other experiments, we found that STAT3 knockdown in neural stem cells, presumed cells of origin of gliomas, also blocked LIF-inhibition of cell proliferation as determined by measuring the fraction of cells incorporating bromodeoxyuridine (BrdU) (FIG. 2E).

The results indicate that LIF-induction of endogenous STAT3 in PTEN expressing glioma cells and in presumed cells of origin of gliomas inhibits cell proliferation. These results indicated that the inhibition of the STAT3 signaling pathway in PTEN-deficient glioma cells may serve to promote glioma growth.

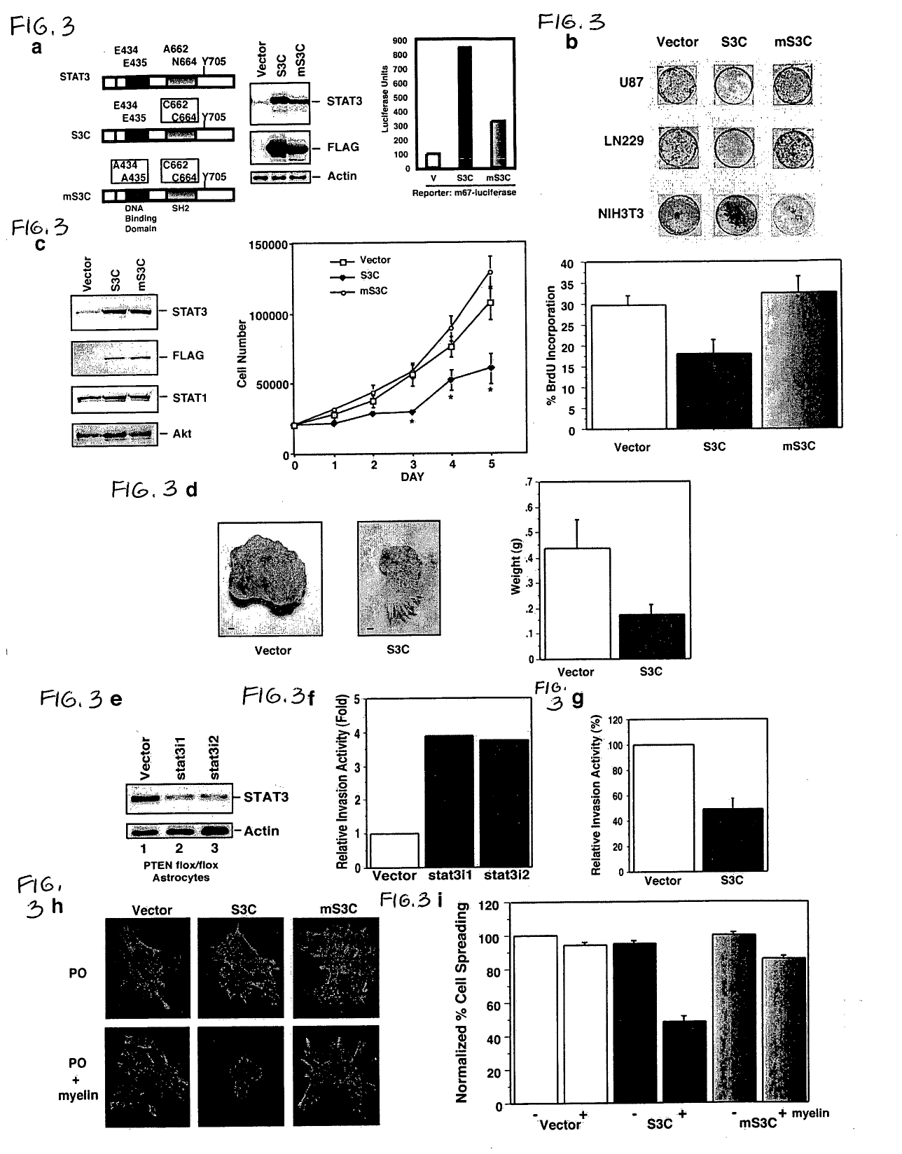

To directly assess the significance of the PTEN loss-triggered suppression of the LIFRβ-STAT3 signaling pathway in glial tumorigenesis, we determined the effect of reactivation of STAT3 in PTEN-deficient astrocytes and glioma cells. Using a retroviral approach, we expressed in these cells a constitutively active form of STAT3 (S3C) that forms dimmers independently of Tyr 705 phosphorylation₂₁. We generated a mutant form of S3C in which the DNA binding region of S3C was mutated (mS3C) (FIG. 3A). When expressed in 293T cells, S3C but not mS3C robustly induced transcription of a reporter gene driven by STAT binding sites suggesting that mS3C serves as a transcriptionally-impaired control mutant of S3C (FIG. 3A).

In colony suppression assays, S3C but not mS3C dramatically inhibited the growth of the PTEN-deficient glioma cell lines U87 and A172 (FIG. 3B). As expected based on the function of endogenous STAT3 in PTEN-expressing glioma cells (FIGS. 2C and 2D), S3C but not mS3C also inhibited the growth of the PTEN-expressing glioma cell line LN229 (FIG. 3B). However, the STAT3-inhibition of colony formation was specific to glioma cells as S3C failed to inhibit the formation of NIH-3T3 cell colonies (FIG. 3B). To determine how S3C inhibits glioma growth, we stably expressed S3C or mS3C in the U87 glioblastoma cell line. In cell growth assays, S3C— but not mS3C-expressing U87 glioblastoma cells increased in number at a significantly lower rate than control vector-infected U87 cells (FIG. 3C). There was no evidence of augmented cell death in S3C-expressing glioblastoma cells. By contrast, the rate of BrdU incorporation in S3C— but not mS3C-expressing glioblastoma cells was reduced when compared to control vector infected cells (FIG. 3C). Our results indicated that activated STAT3 specifically inhibits the proliferation of glioma cells.

To establish that STAT3 inhibits glioma growth, we tested the effect of activated STAT3 on PTEN deficiency's ability to promote glial tumorigenesis in vivo. PTEN−/− mouse astrocytes form tumors when injected subcutaneously into SCID mice. Strikingly, we found that the expression of S3C in PTEN−/− mouse astrocytes significantly reduced the size of astrocytomas in SCID mice (FIG. 3D). These findings indicate that activated STAT3 suppresses glioma growth, and inhibition of the STAT3 signaling pathway in astrocytes upon PTEN deficiency reduces the tumor suppressive properties of STAT3 in these cells.

The function of STAT3 as a suppressor of glioma cell proliferation and tumor growth stands in stark contrast to STAT3's oncogenic behavior outside the nervous system. This finding and the observation that PTEN deficiency suppresses the LIFRβ-STAT3 signaling pathway specifically in astrocytes led us to consider the possibility that STAT3 might also control the unique property of glioma cell invasiveness. We first determined if endogenous STAT3 contributes to glial cell invasiveness. We induced lentiviral-mediated STAT3 RNA interference in wild type PTEN-expressing astrocytes and subjected these cells to a matrigel invasion assay. Knockdown of endogenous STAT3 in the immortalized astrocytes was confirmed by immunoblotting (FIG. 3E). We found that the stat3 hairpin RNA-expressing astrocytes displayed a higher level of invasiveness through matrigel than control-infected astrocytes (FIG. 3F). These results suggest that endogenous STAT3 inhibits glial cell invasiveness.

We next determined if reactivation of STAT3 in PTEN-deficient glioma cells alters their invasiveness. In matrigel invasion assays, U87 glioma cells very efficiently invaded the matrigel layer (FIG. 3G). However, expression of activated STAT3 (S3C) in U87 glioma cells significantly reduced the ability of these cells to invade matrigel (FIG. 3G). These results indicate that in addition to suppressing glioma cell proliferation, STAT3 inhibits glioma cell invasiveness.

Invasiveness of tumor cells was evaluated as follows. To determine if STAT3 regulates glioma cell behavior on a substrate that is relevant to the selective property of gliomas to invade brain tissue along white matter tracts, we tested the effect of S3C on the ability of glioma cells to adhere and spread on adult rat brain myelin. Unlike NIH-3T3 cells which spread on polynomithine but not on myelin, U87 glioblastoma cells spread equally well on polyomithine or myelin substrate forming actin stress fibers around the cell cortex (FIG. 3H). The selective ability of glioblastoma cells but not other tumor or immortalized cells to spread on myelin substrate provides a powerful assay that may reflect the propensity of malignant gliomas to migrate along brain white matter tracts. Strikingly, by contrast to parental U87 glioblastoma cells or those infected with the control GFP virus or the mS3C virus, the majority of S3C-expressing U87 glioblastoma cells spread on polyomithine substrate but failed to spread or form stress fibers in their periphery on myelin (FIGS. 3I and 3J). Thus, activated STAT3 specifically inhibits glioma cell adhesion and spreading on myelin.

Having characterized the functional effects of STAT3 on glioma cell proliferation and invasiveness, we next set out to elucidate the mechanism by which STAT3 mediates these biological effects. Since STAT3′s ability to inhibit glioma cell proliferation and invasiveness correlated with its function as a transcription factor, experiments were carried out to determine whether activated STAT3 triggers changes in the expression of specific genes that mediate STAT3′s glioma suppressive properties.

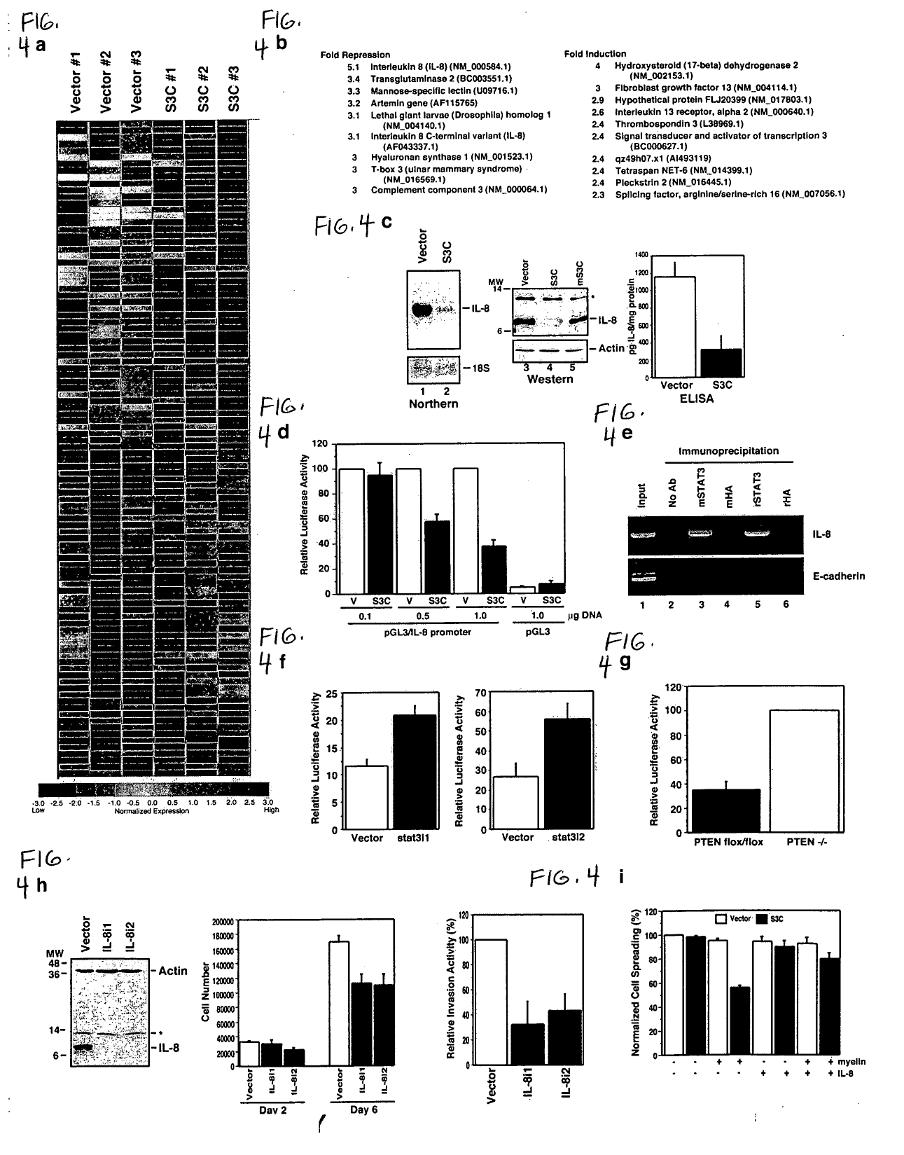

S3C-expressing and control GFP-expressing U87 glioblastoma cells were subjected to microarray analyses using Affymetrix chips. In these analyses, the most profound alteration was a 5-fold repression in the expression of the gene encoding interleukin-8 (IL-8) in S3Cexpressing cells compared to control cells (FIGS. 4A and 4B). Tables 1 and 2 list the genes tested in the microarray analyses, ranked according to fold change, that were repressed or induced upon S3C expression. Tables 1 and 2 list the genes tested in the microarray analyses, ranked according to fold change, that were repressed or induced upon S3C expression.

TABLE 1
Genes Decreased in S3C compared to Vector

| | | Fold |
|---|---|---|
| Name | Accession No. | Decrease |
| Interleukin 8 | NM_000584.1 | 5.1 |
| Transglutaminase 2 | BC003551.1 | 3.4 |
| Mannose-specific lectin | U09716.1 | 3.3 |
| Artemin gene | AF115765 | 3.2 |
| Lethal giant larvae (Drosophila) | NM_004140.1 | 3.1 |
| homolog 1 | | |
| Interleukin 8 C-terminal variant | AF043337.1 | 3.1 |
| Hyaluronan synthase 1 (HAS1) | NM_001523.1 | 3 |
| T-box 3 (ulnar mammary syndrome) | NM_016569.1 | 3 |
| Complement component 3 | NM_000064.1 | 3 |
| Leukemia inhibitory factor | NM_002309.2 | 3 |
| Protein tyrosine phosphatase, | NM_002840 | 2.8 |
| receptor type, F | | |
| Lectin, mannose-binding, 1 | NM_005570 | 2.7 |
| Thrombospondin 1 | NM_003246 | 2.7 |
| Lethal giant larvae (Drosophila) | NM_004140 | 2.6 |
| homolog 1 | | |
| Tumor protein D52 | NM_005079 | 2.6 |
| Melanoma antigen, family C, 1 | NM_005462.1 | 2.6 |
| Chondroitin beta1,4N- | NM_018371 | 2.6 |
| acetylgalactosaminyltransferase | | |
| Leukocyte-derived arginine | NM_022350 | 2.5 |
| aminopeptidase | | |
| Tissue inhibitor of | NM_000362 | 2.5 |
| metalloproteinase 3 | | |
| Homo sapiens cDNA 7q53a12.x1 | BF433429 | 2.5 |
| ATP-binding cassette, sub-family | NM_020037 | 2.5 |
| C (CFTRMRP), member 3 | | |
| Tissue inhibitor of | NM_000362.3 | 2.5 |
| metalloproteinase 3 | | |
| DEAD-box protein p72 | U59321.1 | 2.4 |
| Metallothionein 1K | NM_176870 | 2.4 |
| Protein inhibitor of activated | AF077953.1 | 2.4 |
| STAT protein PIASx-alpha | | |
| Homeo box A4 (HOXA4) | NM_002141 | 2.4 |
| Carbohydrate (keratan sulfate | NM_003654 | 2.4 |
| Gal-6) sulfotransferase 1 | | |
| Insulin receptor substrate-2 | AF073310 | 2.4 |
| (IRS2) | | |
| Tumor protein D52 | NM_005079.1 | 2.4 |
| Chromosome 21 open reading | NM_021941.1 | 2.3 |
| frame 97 | | |
| Homo sapiens cDNA b24g09.x1 | AI307760 | 2.3 |
| Immunoglobulin superfamily, | NM_014333 | 2.3 |
| member 4 | | |
| Tissue inhibitor of | U67195 | 2.3 |
| metalloproteinase-3 | | |
| G protein-coupled receptor 56 | NM_005682 | 2.3 |
| Tumor protein D52 | NM_005079.1 | 2.3 |
| cDNA FLJ13511, highly similar | AK023573.1 | 2.2 |
| to Homo sapiens 7h3 protein | | |
| Hypothetical protein FLJ35036 | NM_024724.1 | 2.2 |
| NECDIN related protein | U35139 | 2.2 |
| HERV-H LTR-associating 1 | NM_005712.1 | 2.2 |
| Cholesterol 25-hydroxylase | NM_003956 | 2.2 |
| Homo sapines cDNA wg58b07.x1, | AI760130 | 2.2 |
| similar to protein phosphatase 2 | | |
| Zinc finger protein 175 | NM_007147 | 2.2 |
| Sequence from clone RP5-1054A22 | AL031651 | 2.2 |
| on chromosome 20q11.22-12 | | |
| Matrix metalloproteinase 3 | NM_002422 | 2.2 |
| Tissue inhibitor of | U67195 | 2.2 |
| metalloproteinase 3 | | |
| Transglutaminase | M98478 | 2.2 |
| DEAD (Asp-Glu-Ala-Asp) box | NM_030881 | 2.2 |
| polypeptide 17 | | |
| Homo sapiens cDNA DKFZp566A0946 | AL050069.1 | 2.2 |
| Homo sapiens cDNA clone IMAGE: | BG402105 | 2.1 |
| 4593682 | | |
| Formyl peptide receptor 1 | NM_002029 | 2.1 |

TABLE 2
Genes Increased in S3C Compared to Vector

| | Accession | Fold |
|---|---|---|
| Name | No. | Increase |
| Hydroxysteroid (17-beta) | NM_002153.1 | 4 |
| dehydrogenase 2 | | |
| Fibroblast growth factor 13 | NM_004114.1 | 3 |
| Hypothetical protein FLJ20399 | NM_017803.1 | 2.9 |
| Interleukin 13 receptor, alpha 2 | NM_000640.1 | 2.6 |
| Thrombospondin 3 | L38969.1 | 2.4 |
| Signal transducer and activator | BC000627.1 | 2.4 |
| of transcription 3 | | |
| qz49h07.x1 | AI493119 | 2.4 |
| Tetraspan NET-6 | NM_014399.1 | 2.4 |
| Pleckstrin 2 | NM_016445.1 | 2.4 |
| Splicing factor, | NM_007056.1 | 2.3 |
| arginine/serine-rich 16 | | |
| Alpha-ketoglutarate | S72422 | 2.3 |
| dehydrogenase complex | | |
| Homo sapiens cDNA DKFZp434G1835 | AL117508.1 | 2.3 |
| Homo sapiens chromosome 14 open | NM_014169.1 | 2.2 |
| reading frame 123 | | |
| Forkhead box G1B | NM_005249.1 | 2.2 |
| Ankyrin repeat and SOCS box- | BC001719.1 | 2.2 |
| containing 6 | | |
| Nicotinamide nucleotide | NM_012343.1 | 2.1 |
| transhydrogenase | | |
| Neuronal protein (NP25) | NM_013259.1 | 2.1 |
| Amyloid A4 protein | X06989.1 | 2.1 |
| HLA class II region expressed | NM_014260.1 | 2.1 |
| gene KE2 | | |
| Ribonuclease, RNase A family, | NM_002933 | 2.1 |
| 1 (pancreatic) | | |
| Homo sapiens clone 127 opioid | AF172449.1 | 2.1 |
| growth factor receptor | | |
| Polymerase (DNA directed), delta | NM_002691 | 2.1 |
| 1, catalytic subunit (125 kD) | | |
| U2(RNU2) small nuclear RNA | NM_006758 | 2.1 |
| auxiliary factor 1 | | |
| Homo sapiens C1orf24 mRNA | AF288391.1 | 2.1 |
| SET and MYND domain containing 3 | NM_022743 | 2.1 |
| Homo sapiens cDNA oa80c10.s1 | AA769006 | 2.1 |
| Homo sapiens cDNA FLJ11118 | AK001980.1 | 2 |
| Actin, alpha 2, smooth muscle, | NM_001613 | 2 |
| aorta | | |
| Retinaldehyde-binding protein 1 | NM_000326 | 2 |
| HNOEL-iso protein | NM_020190 | 2 |
| Brain and acute leukemia, | NM_024812.1 | 2 |
| cytoplasmic | | |
| DEAD (Asp-Glu-Ala-Asp) box | NM_004398 | 2 |
| polypeptide 10 | | |
| MHC HLA-DQ beta mRNA | M32577 | 2 |
| Calcium-regulated heat-stable | NM_014316 | 2 |
| protein 1 (24 kD) | | |
| Hypothetical protein FLJ23311 | NM_024680 | 2 |
| Homo sapiens cDNA zr35c05.s1 | AA195017 | 2 |
| Transgelin | NM_003186 | 2 |
| Kelch repeat and BTB (POZ) | NM_018095 | 2 |
| domain containing 4 | | |
| SKB1 homolog (S. pombe) | NM_006109 | 2 |
| Bromodomain adjacent to zinc | NM_013448 | 2 |
| finger domain, 1A | | |
| Matrix metalloproteinase 14 | NM_004995 | 2 |
| (membrane-inserted) | | |
| Homo sapiens cDNA yj89b06.s1 | R72286 | 1.9 |
| Transcription factor AP-4 | NM_003223 | 1.9 |
| (activating enhancer-binding | | |
| protein 4) | | |
| Sorting nexin 6 | NM_021249 | 1.9 |
| Fas (TNFRSF6)-associated via | NM_003824 | 1.9 |
| death domain | | |
| Hypothetical protein FLJ10948 | NM_018281 | 1.9 |
| Nephropontin precursor | M83248 | 1.9 |
| HT021 | NM_020685 | 1.9 |
| Hypothetical protein FLJ10970 | NM_018286 | 1.9 |
| Human DNA sequence from clone | AL008583 | 1.9 |
| RP3-327J16 on chromosome | | |
| 22q12.3-13.2 | | |

The following studies were carried out to address the question of whether the repression of IL-8 mediates STAT3-inhibition of glioma cell proliferation and invasiveness. First, we confirmed the microarray data in Northern analyses in which the amount of IL-8 mRNA was robustly suppressed in S3C-expressing cells (FIG. 4C). Consistent with these results, immunoblotting of U87 glioblastoma cell extracts revealed that IL-8 protein was significantly downregulated upon expression of S3C. By contrast, IL-8 protein was reduced to a lesser extent in mS3C-expressing cells suggesting that the transcriptional function of STAT3 is required for regulation of IL-8 expression (FIG. 4C). The secretion of mature soluble IL-8 in the medium by these glioma cells correlated tightly with the results of Northern and Western analyses (FIG. 4C). The following studies were carried out to address whether STAT3 inhibits the expression of IL-8 in glioma cells.

In transient expression assays in astrocytes, S3C expression potently diminished IL-8 promoter-mediated transcription (FIG. 4D). By chromatin immunoprecipitation analyses of S3Cexpressing U87 glioblastoma cells, activated STAT3 was specifically bound to the endogenous IL-8 promoter in these cells (FIG. 4E). These findings indicated that IL-8 is a direct gene target of STAT3 and that activated STAT3 represses IL-8 transcription.

To determine the role of endogenous STAT3 in IL-8 transcription, we induced lentivirally-mediated STAT3 RNA interference in the wild type PTEN-expressing astrocytes. STAT3 knockdown led to an enhancement of IL-8 promoter-mediated transcription suggesting that endogenous STAT3 represses the IL-8 promoter in wild type PTEN-expressing astrocytes (FIG. 4F). A prediction of this result is that PTEN loss, which suppresses the STAT3 signaling pathway, should derepress the IL-8 promoter. Consistent with this prediction, we found that IL-8 promoter-mediated transcription was robustly stimulated in astrocytes upon PTEN loss (FIG. 4G). Taken together, these results indicated that STAT3 directly represses the IL-8 promoter in astrocytes and that suppression of the STAT3 signaling pathway upon PTEN loss relieves the IL-8 promoter repression.

To determine if the repression of IL-8 contributes to STAT3's ability to inhibit glioma cell proliferation, we used a lentiviral DNA template-based method of RNA interference to knockdown IL-8 in U87 glioblastoma cells (FIG. 4H). Knockdown of endogenous IL-8 in U87 glioblastoma cells by two hairpin RNAs targeting distinct regions of IL-8 significantly reduced cell proliferation (FIG. 4H). IL-8 knockdown also significantly reduced the ability of U87 cells to invade matrigel (FIG. 4H). In other experiments, we determined if IL-8 restores the ability of S3C-expressing U87 glioblastoma cells to spread on myelin substrate. Upon exposure to IL-8, the S3C-expressing U87 glioblastoma cells spread as efficiently as control vector-infected U87 cells on myelin (FIG. 4I). Together, these results indicated that IL-8 is a STAT3-repressed gene that promotes glioma cell proliferation and invasiveness.

The results described herein revealed that PTEN deficiency triggered the inhibition of FOXO3-dependent LIFRβ gene expression and the consequent suppression of the LIFRβ-STAT3 signaling pathway specifically in astrocytes. We found that activation of endogenous STAT3 inhibited wild type PTEN-expressing glioma cell proliferation and invasiveness. Conversely, reactivation of STAT3 in PTEN-deficient astrocytes or glioma cells inhibited proliferation, reversed glioma growth in vivo, and suppressed glioma cell invasiveness and spreading on myelin. The STAT3-inhibition of glioma cell proliferation and invasiveness occurred via STAT3-repression of its novel direct target gene, the chemokine IL-8. Thus, deregulation of the gliogenesis-promoting LIFRβ-STAT3 signaling pathway and upregulation of IL-8 may confer PTEN loss with the ability to stimulate glioma growth and invasiveness.

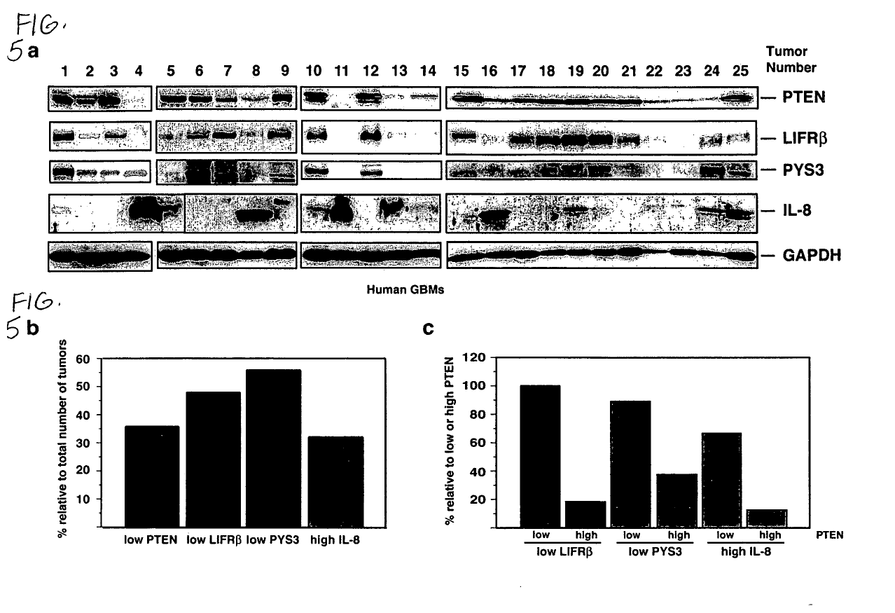

To determine the relevance of our findings in astrocytes and glioblastoma cells to glial tumorigenesis in the human brain, we measured by immunoblotting the levels of PTEN, LIFRβ, phosphorylated STAT3, and IL-8 in 25 specimens of glioblastoma tumors. A large fraction of these tumors displayed low levels of PTEN, low LIFRβ, and low phosphorylated STAT3 (FIG. 5B). Strikingly, we found a direct relationship between PTEN loss and low LIFRβ, and between PTEN loss and low phosphorylated STAT3 levels. Thus, all tumors with low PTEN levels had low levels of LIFRβ (FIGS. 5A-5C). Similarly, 89% of low PTEN-expressing tumors had low levels of phosphorylated STAT3 (FIGS. 5A and 5C). However, only a small fraction of high PTEN-expressing tumors had low levels of LIFRβ and phosphorylated STAT3. In contrast to the direct relationship between low PTEN and low LIFRβ, and between low PTEN and low phosphorylated STAT3, we found an inverse relationship between low PTEN and IL-8 expression. Over 67% of tumors with low PTEN had high levels of IL-8 (FIG. 5A and 5C).

Among tumors with high IL-8 expression, several (tumors 4, 8, 11, 13, and 16) had the striking profile of very low levels of PTEN, LIFRβ, and phosphorylated STAT3, with very high levels of IL-8 expression (FIG. 5A). Together with our studies in astrocytes and glioblastoma cells, these findings indicate that suppression of the LIFRβ-STAT3 signaling pathway and upregulation of IL-8 occurs as a consequence of PTEN deficiency in human glial tumorigenesis.

The data described herein were unexpected and indicate that, while STAT3 harbors an oncogenic role in non-neural tissues, STAT3 exerts tumor suppressive effects in CNS tumor cells such as glial cells. It has also been suggested that STATs may promote glioma tumor cell proliferation and survival, and STAT3 activation has been suggested to occur in glial tumors. However, other studies have provided evidence of the absence of STAT3 activation in gliomas. Our study sheds light on the role of STAT3 in glial tumorigenesis. In contrast to the oncogenic role of STAT3 in non-neural cells, our findings indicate that STAT3 inhibits glioma cell proliferation and growth and that suppression of the STAT3 signaling pathway is critical for the ability of PTEN deficiency to promote glioma cell proliferation. A growth suppressive role of STAT3 in astrocytes is consistent with the function of STAT3 as a transcription factor dedicated to the differentiation of neural stem cells into astrocytes during normal brain development.

Inhibition of the gliogenesis-promoting LIFRβ-STAT3 signaling pathway in glioma cells also serves to couple tumor growth to the glioma cell-specific behavior of invasiveness and attachment and spreading on myelin. The specific PTEN loss-triggered suppression of LIFRβ-STAT3 signaling in astrocytes but not in non-neural cells illustrates how interactions between ubiquitously-acting tumor suppressors and developmental signaling pathways implicated in cell differentiation and fate specification may produce tumor-specific pathological behaviors.

The identification of IL-8 as a STAT3-repressed target gene in glioma cells, whose repression mediates STAT3-inhibition of glioma cell proliferation, invasiveness, and spreading on myelin defines a cytokine-chemokine connection. These findings also indicate that STAT3 acts as a transcriptional repressor in CNS tumor cells such as glioma cells.

What is claimed is:

1. A method of reducing the growth or invasiveness of a tumor cell comprising: contacting said tumor cell with an agent that reduces the level or activity of IL-8 or IL-8 receptor.

2. The method of claim 1, wherein said tumor cell is PTEN-deficient.

3. The method of claim 1, wherein said tumor cell is of the central nervous system (CNS).

4. A method of reducing or preventing the growth or invasiveness of a brain tumor in a mammal by administering to said mammal an agent that reduces the level or activity of IL-8 or IL-8 receptor (IL-8R).

5. The method of claim 1, wherein said agent increases the level or activity of STAT 3 in neuronal cells of said mammal.

6. The method of claim 3, wherein said tumor cell is a glial cell.

7. The method of claim 1, wherein said agent is a small molecule inhibitor.

8. The method of claim 7, wherein said small molecule inhibitor is a CXCR1/2 antagonist.

9. The method of claim 8, wherein said CXCR1/2 antagonist is a CXCR1 antagonist or a CXCR2 antagonist.

10. The method of claim 9, wherein said CXCR1 antagonist is repertaxin.

11. The method of claim 1, wherein said tumor cell or tumor is a glioma.

12. The method of claim 11, wherein said glioma is an astrocytoma.

13. The method of claim 12, wherein said astrocytoma is a glioblastoma multiforme.

14. The method of claim 1, wherein said mammal is further administered a second therapeutic regimen.

15. The method of claim 14, wherein said therapeutic regimen is surgery, radiotherapy, or chemotherapy.

16. A method of reducing the growth or invasiveness of a PTEN-deficient tumor cell comprising contacting said tumor cell with an agent that increases the level or activity of STAT 3 in neuronal cells of said mammal.

17. A method of reducing or preventing the growth or invasiveness of a CNS tumor in a mammal by administering to said mammal an agent that increases the level or activity of STAT 3 in neuronal cells of said mammal.

18. The method of claim 14, wherein said neuronal cells are glial cells.

19. The method of claim 14, wherein said agent is a small molecule activator

20. The method of claim 14, wherein said tumor is a glioma.

21. The method of claim 20, wherein said glioma is an astrocytoma.

22. The method of claim 21, wherein said astrocytoma is a glioblasma multiforme.

23. The method of claim 14, wherein said mammal is further administered a second therapeutic regimen.

24. The method of claim 23, wherein said therapeutic regimen is surgery, radiotherapy, or chemotherapy.
